(19) United States
(12) Patent Application Publication
(10) Pub. No.: US 2009/0142311 A1
(43) Pub. Date: Jun. 4, 2009

(54) ENGINEERED INTERVERTEBRAL DISC TISSUE

(76) Inventors: Koichi Masuda, Wilmette, IL (US); Howard S. An, Riverwood, IL (US); Eugene J-M. A. Thonar, Lockport, IL (US)

Correspondence Address: FOLEY & LARDNER LLP, 150 EAST GILMAN STREET, P.O. BOX 1497, MADISON, WI, 53701-1497, US

(21) Appl. No.: 12/356,920

(22) Filed: Jan. 21, 2009

Related U.S. Application Data
(63) Continuation of application No. 11/273,164, filed on Nov. 14, 2005, now abandoned.
Application No. 11/273,164 is a continuation of application No. 10/084,640, filed on Feb. 25, 2002, now abandoned.
(60) Provisional application No. 60/348,111, filed on Nov. 9, 2001.

(51) Int. Cl. A61K 35/12; C12N 5/08; A61P 41/00

(52) U.S. Cl. 424/93.7; 435/384; 435/366

(57) ABSTRACT

Surgically implantable tissue engineered intervertebral disc tissues that effectively replicate the physicochemical properties of the corresponding in vivo tissues are provided. Methods for producing such tissues can involve culturing the intervertebral disc cells to produce cells surrounded by an extracellular matrix and culturing the cells and matrix on a semipermeable membrane to form a cohesive tissue.

### STATEMENT REGARDING CLAIM OF PRIORITY

This application is a continuation of U.S. application Ser. No. 11/273,164, filed Nov. 14, 2005, which application is a continuation of U.S. application Ser. No. 10/084,640, filed Feb. 25, 2002; which application claims the benefit of U.S. Provisional Application Ser. No. 60/348,111, filed Nov. 9, 2001. The entire contents of the aforementioned applications are hereby incorporated by reference in their entireties.

### STATEMENT REGARDING FEDERALLY SPONSORED RESEARCH

This invention was made with Government support under grant No. 2-P50-AR39329 awarded by the National Institutes of Health, National Institute of Arthritis, Musculoskeletal and Skin Diseases and grant No. AG-04736 awarded by the National Institute on Aging. The Government has certain rights in this invention.

### FIELD OF INVENTION

The present invention relates to engineered intervertebral disc tissues and systems and methods to produce and utilize these tissues. More particularly this invention relates to engineered nucleus pulposus and annulus fibrosus tissues, their production and use.

### BACKGROUND OF THE INVENTION

Back pain is the second most common ailment complained about in doctors' offices after the common cold and is responsible for some 100 million lost days of work annually in the United States alone. A major proportion of these back injuries result from damage to the intervertebral discs in the spine. The intervertebral disc has a unique structure, comprised of a tough outer ring called the annulus fibrosus, and a gelatinous inner core called the nucleus pulposus. The annulus fibrosus, along with the endplates of the vertebrae above and below it, contains the nucleus pulposus and resists the deformation of the nucleus pulposus that would otherwise occur under mechanical loading. The unique organization of the disc components confers upon the intervertebral disc the properties of flexibility and resiliency necessary for normal function. When the integrity of the annulus fibrosus is compromised, disc degeneration may ensue or frank herniation of the nucleus pulposus may occur. The loss of integrity of the annulus fibrosus may be part of the pathological process or surgically created, as occurs during removal of the nucleus pulposus in lumbar discectomy. Lumbar disc degeneration and herniation of the nucleus pulposus are also major causes of low back pain and disability. These problems are compounded because intervertebral disc tissue is slow to heal because it does not have a direct supply of blood and must derive its nutrients elsewhere.

No method currently exists for directly treating an annulus fibrosus defect and therefore no treatment exists that truly restores the disc to its pre-injury state. While symptomatic treatments for lumbar disc disease, such as lumbar discectomy and fusion, are available, the injured intervertebral disc is permanently altered in terms of its mechanical load bearing properties. This frequently leads to chronic low back pain and degeneration at other levels of the spine. Likewise, no acceptable method of studying these tissues outside of the body is known because the annulus fibrosus and nucleus pulposus rapidly degrade or change physically or in composition once outside the body.

Thus there continues to be a strong need for engineered tissues that resemble intervertebral disc tissues, not only for the treatment for intervertebral disc defects but also to provide medical insight into the composition and workings of these tissues.

### SUMMARY OF THE INVENTION

One embodiment of the present invention provides engineered intervertebral disc tissues. These cohesive tissues can be made up of greater than or about 80 percent water by weight, between at or about 0.95 and 7.5 μg/mg DNA based on the dry weight of the tissue, between at or about 100 and 350 μg/mg proteoglycan based on tissue dry weight, and between at or about 75 and 450 μg/mg collagen based on tissue dry weight.

Another embodiment of the present invention also provides methods for producing an engineered intervertebral disc tissue. The tissue can be produced by culturing intervertebral disc cells in a medium for an effective amount of time to produce intervertebral disc cells surrounded by a cell-associated matrix and culturing these cells on a semipermeable membrane in the presence of one or more growth factors for a sufficient amount of time to produce a coherent, engineered intervertebral disc tissue. The method can also include one or more steps, such as isolating the intervertebral disc cells prior to culturing them to form extracellular matrix, recovering the intervertebral disc cells surrounded by the cell-associated matrix, removing the engineered intervertebral disc tissue from the semipermeable membrane or implanting the engineered intervertebral disc tissue into an intervertebral disc in vivo.

Objects and advantages of the present invention will become more readily apparent from the following detailed description.

### DETAILED DESCRIPTION OF INVENTION

In one embodiment of the present invention, engineered intervertebral disc tissue (IVD) that physiochemically resembles naturally occurring intervertebral disc tissue is provided. Intervertebral discs separate the spinal vertebrae from one another and act as natural shock absorbers by cushioning impacts and absorbing the stress and strain transmitted to the spinal column. Intervertebral disc tissues are composed of three categories, the end plates, the annulus fibrosus (AF) and the nucleus pulposus (NP). The annulus fibrosus is a tough collagen-fiber composite that has an outer rim of type I collagen fibers surrounding a less dense fibrocartilage and a transitional zone. These collagen fibers are organized as cylindrical layers. In each layer the fibers are parallel to one another, however the fiber orientation between layers varies between 30 and 60 degrees. This organization provides support during torsional, bending and compressive stresses on the spine. The end plates, which are found at the upper and lower surfaces of the disc, work in conjunction with the annulus fibrosus to contain the gel-like matrix of the nucleus pulposus within the intervertebral disc. The nucleus pulposus is made up of a soft matrix of proteoglycans and randomly oriented type II collagen fibers in water. The proteoglycan and water content are greatest at the center of the disc and decrease toward the disc periphery. Tissues that effectively mimic these structures can be produced according to the methods discussed herein.

The present invention also provides methods and kits for producing these tissues and methods for repairing intervertebral disc defects using these tissues. Generally, in one method intervertebral disc cells are isolated and cultured to produce cells with a cell-associated matrix. These cells and their surrounding cell-associated matrix are then cultured on a semi-permeable membrane in the presence of one or more growth factors to produce an engineered intervertebral disc tissue. One such method for culturing an engineered tissue can be found in U.S. Pat. No. 6,197,061 entitled “In vitro Production of Transplantable Cartilage Tissue, Cohesive Cartilage Produced Thereby, and Method for the Surgical Repair of Cartilage Damage” issued to Masuda et al., the contents of which are explicitly incorporated herein. The methods disclosed herein can include any, some or all of the disclosed steps.

### Isolation of Intervertebral Disc Cells

Intervertebral disc cells useful in the present methods can be obtained and/or isolated from essentially any intervertebral disc tissue, such as nucleus pulposus or annulus fibrosus. Preferably, cells are obtained from only one type of intervertebral disc source and are not mixed with intervertebral disc cells of another type, i.e. obtained nucleus pulposus cells are essentially free of annulus fibrosus cells. However, the present invention contemplates obtaining a homogeneous sample of intervertebral disc cells containing end plate, nucleus pulposus, annulus fibrosus cells or combinations thereof. Also, the composite graft using two different tissues can be engineered by adding two cell types by layer or adding the nucleus pulposus cells in the center of annulus tissues to mimic the original intervertebral disc tissue.

Alternatively, cells can be isolated from bone marrow. See for example, U.S. Pat. Nos. 5,197,985 and 4,642,120, and Wakitani et al. (1994) J. bone Joint Surg. 76:579-591, the disclosures of which are incorporated by reference herein. Intervertebral disc cells can also be derived from stem cells.

Suitable intervertebral disc cells can be isolated from any suitable mammalian source organism, including, without limitation, human, orangutan, monkey, chimpanzee, dog, cat, rat, mouse, horse, cow, pig, and the like. Intervertebral disc cells can be either isolated from sources having normal intervertebral disc tissue or tissue which is known to be defective in some manner, such as having a genetic defect.

Intervertebral disc cells used for preparation of the in vitro cell culture device of the present invention can be isolated by any suitable method. Various starting materials and methods for cell isolation are known (see generally, Freshney, Culture of Animal Cells: A Manual of Basic Techniques, 2d ed., A. R. Liss Inc., New York, pp 137-168 (1987); Klagsburn, “Large Scale Preparation of Chondrocytes,” Methods Enzymol. 58:560-564 (1979); Shinmei, M., T. Kikuchi, et al. (1988). “The role of interleukin-1 on proteoglycan metabolism of rabbit annulus fibrosus cells cultured in vitro.” Spine 13(11): 1284-90; Maldonado, B. A. and T. R. Oegema, Jr. (1992). “Initial characterization of the metabolism of intervertebral disc cells encapsulated in microspheres.” J Orthop Res 10(5): 677-90.

If the starting material is a tissue in which intervertebral disc cells are essentially the only cell type present, e.g., intervertebral disc tissue, the cells can be obtained directly by conventional enzymatic digestion and tissue culture methods. Alternatively, the cells can be isolated from other cell types present in the starting material. One known method for cell isolation includes differential adhesion to plastic tissue culture vessels. In a second method, antibodies that bind to intervertebral disc cell surface markers can be coated on tissue culture plates and then used selectively to bind intervertebral disc cells from a heterogeneous cell population. In a third method, fluorescence activated cell sorting (FACS) using intervertebral disc-specific antibodies is used to isolate cells. In a fourth method, cells are isolated on the basis of their buoyant density, by centrifugation through a density gradient such as Ficoll.

It can be desirable in certain circumstance to utilize intervertebral disc stem cells rather than differentiated intervertebral disc cells. Examples of tissues from which stem cells for differentiation, or differentiated cells suitable for transdifferentiation, can be isolated include placenta, umbilical cord, bone marrow, skin, muscle, periosteum, or perichondrium. Cells can be isolated from these tissues through an explant culture and/or enzymatic digestion of surrounding matrix using conventional methods.

### Culture in Medium for the Production of Cell-Associated Matrix

Isolated intervertebral disc cells are suspended at a density of preferably at least about 10⁴ cells/ml in an appropriate medium, such as agarose or sodium alginate. The cells are preferably cultured under conditions effective for maintaining their phenotypic conformation conducive to the production of a cell-associated matrix similar to that found in vivo. Preferably, intervertebral disc cells are cultured in alginate for at least about five days to allow for formation of a cell-associated matrix. The media within which the intervertebral disc cells are cultured can contain a stimulatory agent, such as fetal bovine serum, to enhance the production of the cell-associated matrix.

The beads containing intervertebral disc cells are cultured in a growth medium, such as equal parts of Dulbecco's modified Eagle medium and Ham's F12 medium containing 20% fetal bovine serum (Hyclone, Logan, Utah), about 25 μg/ml ascorbate and antibiotic, such as 50 μg/ml gentamicin (Gibco). In an alternative approach, the beads are cultured in a closed chamber that allows for continuous pumping of medium. Preferably, the medium contains fetal bovine serum containing endogenous insulin-like growth factor-1 at a concentration of at least about 10 ng/ml. In this usage, fetal bovine serum can also be considered a growth factor. Several serum free culture media such as HL-1™, PC-1™ and UltraCulture™ (BioWhittaker) can be used in place of fetal bovine serum. In an alternative aspect of the invention, the culture medium for the intervertebral disc cells can further include exogenously added specific growth factors. Suitable growth factors that can be exogenously added to the medium to maximally stimulate formation of the cell-associated matrix include but are not limited to osteogenic protein-1 (OP-1), bone morphogenic protein-2 and other bone morphogenetic proteins, cartilage-derived morphogenetic protein, platelet-derived growth factor, fibroblast growth factor, transforming growth factor beta, and insulin-like growth factor. The addition of specific growth factors, for example those not already present in fetal bovine serum, such as osteogenic protein-1, can act as an effective stimulator of matrix formation. In this aspect of the invention, growth factor is added to the medium in an amount to near-maximally stimulate formation of the cell-associated matrix, which is dependent on the type of cells stimulated. In the case of BMP4 or OP-1, typically 50 ng to 200 ng/ml can be used.

Preferably, amplification of intervertebral disc cells in the growth medium does not induce loss of the specific intervertebral disc cell phenotype, as occurs when amplification is performed in monolayer culture. A phenotypically stable intervertebral disc cell can also retain the ability to effectively incorporate the major macromolecules into a intervertebral disc tissue matrix.

Intervertebral Disc Cells with Cell-Associated Matrix

Culture of intervertebral disc cells in alginate results in the production of an extracellular matrix (ECM) that is organized into two compartments: (i) a cell-associated matrix compartment that metabolically resembles the pericellular and territorial matrices of native tissues, and (ii) a further removed matrix compartment that metabolically resembles the interterritorial matrix of native tissue.

Preferably, the cell-associated matrix compartment of the ECM produced during culture in alginate includes proteoglycan, primarily aggrecan, collagen and hyaluronan. Collagen type, such as I or II, can vary in the tissue depending upon the intervertebral disc tissue which the engineered tissue simulates. For example, the main type of collagen in annulus fibrosus is type I, whereas nucleus pulposus contains primarily type II collagen. The present intervertebral disc tissues can also contain minor amounts of other collagens, for example types VI, IX, or XI, and proteoglycans, such as decorin, biglycan and fibromodulin.

Recovery of Intervertebral Disc Cells with Their Cell-Associated Matrix

Recovery of intervertebral disc cells with their cell-associated matrix can be accomplished by solubilizing alginate beads after a sufficient culture period. Alginate beads are first solubilized using known techniques, such as chelation. The resulting cell suspension then is centrifuged, separating the cells with their cell-associated matrix in the pellet from the components of the further removed matrix in the supernatant.

Culturing the Intervertebral Disc Cells with Their Cell-Associated Matrix on a Semipermeable Membrane

In this aspect of the invention, the intervertebral disc cells with their cell-associated matrix isolated as described above, are further cultured on a semipermeable membrane. Preferably, a cell culture insert is placed into a plastic support frame and culture medium flows around the cell culture insert. In this aspect, the cell culture insert includes a semipermeable membrane. The semipermeable membrane allows medium to flow into the cell culture insert in an amount effective for completely immersing the intervertebral disc cells and their cell-associated matrix.

Preferably, the semipermeable membrane allows the intervertebral disc cells to have continuous access to nutrients while allowing the diffusion of waste products from the vicinity of the cells. In this aspect, the membrane should have a pore size effective to prevent migration of intervertebral disc cells through the pores and subsequent anchoring to the membrane, preferably not more than about 5 microns. Further, the membrane utilized should have a pore density effective for providing the membrane with sufficient strength so that it can be removed from its culture frame without curling, and with sufficient strength such that the tissue on the membrane can be manipulated and cut to its desired size. Preferably the membrane should have a pore density of at least about 8×10⁵ pores/cm². The membrane can be made of any material suitable for use in culture. Examples of suitable membrane systems include but are not restricted to: (i) Falcon Cell Culture Insert [Polyethylene terephthalate (PET) membrane, pore size 0.4 to 3 microns, diameter 12 to 25 mm]; (ii) Coaster Transwell Plate [Polycarbonate membranes, pore size, 0.1 to 5.0 microns, diameter 12 to 24.5 mm]; (iii) Nunc Tissue Culture Insert (Polycarbonate Membrane Insert: pore size, 0.4 to 3.0 microns, diameter 10 mm to 25 mm); Millicell Culture Plate Insert [PTFE (polytetrafluoroethylene) membrane, polycarbonate, pore size 0.4 to 3.0 microns, diameter 27 mm].

Culture times will generally be at least about 3 days under standard culture conditions. Culture times can be increased in order to produce intervertebral disc tissue that more effectively mimics mature intervertebral disc tissue. Partial inhibition of matrix maturation prior to implantation can be important in providing a matrix that is not as stiff as mature intervertebral disc tissue, but which has enough tensile strength to retain its shape and structure during handling.

The relative proportions of each component in the cell-associated matrix vary depending on the length of time in culture. Further, the molecular composition of the cell-associated matrix (around each cell) and further removed matrix (between the cells) can be altered by specific modifications of the culture conditions. These modifications involve the physical arrangement of the culture system and application of various growth factors. Manipulation of matrix production and organization are central to the engineering of intervertebral disc tissue in vitro for surgical treatment of intervertebral disc defects.

For example, the mechanical properties and histological content of the intervertebral disc tissue matrix can be controlled by increasing or decreasing the amount of time that the intervertebral disc tissue is cultured on the membrane. Preferably, the contents of collagen and of the pyridinoline crosslinks of collagen increase with time of culture. By keeping the length of the culture period relatively short, the collagen fibrils in the cell-associated matrix do not become overly crosslinked. A tissue that has good functional properties but is relatively deficient in crosslinks is easier to manipulate, surgically implant and is likely more readily integrated in vivo than tissue with higher amounts of cross-linking. Tissues with higher amounts of crosslinking can be desired when more mature intervertebral disc tissue is sought to be simulated. Longer culture time generally results in increased crosslink densities, decreased DNA content (indicative of cellularity) per dry weight of the tissue, increased collagen content, increased hyaluronan content, decreased proteoglycan content per collagen (or relative PG content to collagen) in annulus fibrosus tissue and increased proteoglycan content per collagen in nucleus pulposus tissue.

### Intervertebral Disc Tissue

Preferably, the intervertebral disc tissue of the present invention is made up of greater than or about 80 percent water by weight, between at or about 0.95 and 7.5 μg/mg DNA, between at or about 100 and 350 μg/mg proteoglycan, between at or about 1.5 and 3.0 μg/mg hyaluronan and between at or about 75 and 450 μg/mg collagen. The DNA, proteoglycan, hyaluronan and collagen amounts are based on the dry weight of the engineered tissue. In one embodiment the engineered tissue replicates a more immature nucleus pulposus tissue: the DNA content of the tissue is between about 2.7 and 4.5 μg/mg, the proteoglycan content of the tissue is between at or about 100 and 225 μg/mg, the collagen content of the tissue is between at or about 75 and 200 μg/mg and a majority of the collagen is type II collagen. Where an engineered nucleus pulposus tissue is produced, preferably the intervertebral disc cells are derived from nucleus pulposus tissue. In another embodiment the engineered intervertebral disc tissue replicates a more mature nucleus pulposus tissue wherein the DNA content of the tissue is between at or about 0.95 and 1.25 μg/mg, the proteoglycan content of the tissue is between at or about 250 and 350 μg/mg, the collagen content of the tissue is between at or about 325 and 450 μg/mg and further wherein a majority of the collagen is type II collagen. In another embodiment the engineered intervertebral disc tissue replicates an immature annulus fibrosus tissue in which the DNA content of the tissue is between at or about 3 and 5.8 μg/mg, the proteoglycan content of the tissue is between at or about 100 and 200 μg/mg, the collagen content of the tissue is between at or about 100 and 300 μg/mg and further wherein a majority of the collagen is type I collagen. Where an engineered annulus fibrosus tissue is produced, preferably the intervertebral disc cells are derived from annulus fibrosus tissue.

The engineered intervertebral disc tissue used in the present methods closely resembles naturally occurring intervertebral disc tissue in its physicochemical properties in a short period of time. It is also preferable to remove the engineered tissue from the semipermeable membrane, especially prior to surgical implantation in vivo.

Once obtained, the engineered intervertebral disc tissue can be surgically implanted into an intervertebral disc defect. Desirably, engineered tissue can be transplanted into any suitable defect, including annulus fibrosus tears, nucleus pulposus degeneration or herniation. Preferably, as will be understood by one skilled in the art, when the defect to be repaired occurs in the annulus fibrosus engineered tissue resembling the annulus fibrosus will be utilized whereas when the defect involves nucleus pulposus damage engineered nucleus pulposus tissue will be used. When complex defects or injuries involve both the annulus fibrosus and nucleus pulposus, engineered tissues corresponding to both of these can be used. It is also preferred that the implanted intervertebral disc tissue be an autograft, however, the tissue can also be a suitable allograft or even a xenograft.

Implantation of the engineered tissue can also be accompanied by the administration of other therapeutic molecules, such as growth factors, immune response modulators and the like. In one preferred embodiment, growth factor OP-1 is administered to the defect site at or about the time of the engineered tissue transplantation to promote integration of the engineered tissue and healing of the intervertebral disc defect. Continued administration of the growth factors can also accompany tissue transplantation.

The present culture system can also be used to mimic different pathological states in intervertebral disc tissue, including physical injury and disease states, such as disc degeneration and herniation. According to this embodiment, intervertebral disc tissue is cultured and then either artificially injured, such as by physically cutting or tearing the engineered tissue, or treated with factors, such as inflammatory mediators and matrix degrading compounds, known to cause the progression of disease states. The engineered tissue mimicking a pathological state can then be treated with one or more test agents as described above to determine the effect the test agent has on the pathological state. In this embodiment, as in others, it may be desirable to isolate intervertebral disc cells that are known to have a certain defect, such as a genetic defect.

The cells at any stage described above or tissue produced herein can also be transfected with exogenous DNA. In this manner the cells or tissues can be stimulated to produce additional molecules, such as growth factor OP-1, that can further enhance the matrix production, therapeutic efficacy or implantability of the cells or tissues. Several methods for transfecting cells with DNA are known in the art and include viral mediated transfection, plasmid transfection, cell fusion. microinjection or liposome mediated transfection. Preferably, however, the present cells are tissues are transfected with a “gene gun” which can be either particle or non-particle dependent for transfer of the genetic material. Examples of suitable technology for such transfections include those disclosed in U.S. Pat. Nos. 4,945,050 and 6,093,557. According to this aspect of the invention any gene or genetic material can be introduced in to the cells or tissue. Preferably, the genetic material encodes for the production of one or more growth factors, such as OP-1, which can help promote the integration of the engineered tissue and repair of the natural, damaged tissue. The transfected gene product can be inducible as is well known in the art. Depending upon the method used transfection can occur either in vitro, in vivo or both.

The present invention also provides kits for carrying out the methods described herein. In one embodiment, the kit is made up of instructions for carrying out any of the methods described herein. The instructions can be provided in any intelligible form through a tangible medium, such as printed on paper, computer readable media, or the like. The present kits can also include one or more reagents, buffers, culture media, culture media supplements, growth factors, semipermeable membranes, enzymes capable of degrading the engineered tissue, antibodies for labeling a specific component of the intervertebral disc tissue, chromatic or fluorescent dyes for staining or labeling a specific component of the tissue, radioactive isotopes for labeling specific components of the tissue, and/or disposable lab equipment, such as multi-well plates in order to readily facilitate implementation of the present methods. Examples of such kit components can be found in the examples set out below. Components of the tissue to be stained or labeled can include a fragment of the matrix cleaved by enzymatic action, which may or may not be released into the surrounding media.

This invention is further illustrated by the following non-limiting examples.

### EXAMPLES

### Example 1

### Production and Characterization of Intervertebral Disc Tissue

### Cell Isolation and ARC Culture Method

Bovine IVD cells from the tails of 14-18 month old steer were isolated by sequential enzyme digestion. Chiba et al. Spine, 22:2285, 1997. The ARC method was then used as follows to form discs in vitro. Annulus fibrosus [AF] cells and nucleus pulposus [NP] cells were separately cultured in beads of 1.2% low viscosity alginate (Keltone LV, Kelco) at 4 million cells/ml using daily changes of DMEM/F12 medium containing 20% FBS+OP-1 (200 ng/ml), 25 μg/ml ascorbate and 10 μg/ml gentamicin. The cells with their cell-associated matrix were recovered by centrifugation of alginate beads solubilized in the presence of sodium citrate after 10 days of culture in alginate. The pelleted cells were resuspended in complete medium containing 20% FBS and 200 ng/ml of OP-1, seeded onto a tissue culture insert with a porous membrane (Costar, Transwell: 0.4 μm pore size, 10 mm diameter) and maintained in daily changes of the same medium for up to 4 weeks.

### Characterization of Engineered Tissues In Vitro

After 2 and 4 weeks, the de novo formed tissue was separated in each case from the porous membrane; the weights (dry and wet) were measured and the tissue was subjected to biochemical analyses. Each tissue was also examined histologically. The contents of sulfated PG and DNA were measured by the DMMB method and Hoechst 33258-dye method, respectively. The content of collagen was measured by reverse-phase high-performance liquid chromatography. Compressive and tensile testing were performed to determine the equilibrium compressive modulus, HA0, the hydraulic permeability at 15% of strain, kp15, and the peak tensile stress σₘₐₓ. The data were analyzed statistically using ANOVA.

After 2 weeks, tissues engineered from NP and AF cells had a disk-like structure and were easy to separate from the membrane. The presence of OP-1, at a concentration of 200 ng/ml, stimulated the formation of cohesive discs. Both the wet and dry weights and also the thickness of the NP discs were significantly higher than those of AF discs (Table 1). The water content of NP discs was also significantly higher than those of AF discs (Table 1). Significant PG accumulation was observed in both NP and AF discs, but especially in the former (p<0.01) (FIG. 1A). On the other hand, the collagen content of the AF discs was greater than that of NP discs (p<0.01) (FIG. 1B). H_{A0} and σₘₐₓ varied significantly with cell type (each p<0.01) but not culture duration (p=0.47 and 0.17, respectively). H_{A0} and σₘₐₓ of AF tissue were significantly higher (+170% and +270%, respectively) than those of NP tissue (Table 1). kp15 was lower for AF discs than NP discs (p<0.05), and increased with culture duration (p<0.05), without an interactive effect (p=0.75).

These results demonstrate the present methods can be used to form a disc-shaped tissue by IVD cells. Importantly, the collagen content was higher and the ratio of PG/collagen was lower in the AF than in the NP tissue, consistent with the observation that AF cells form a more fibrous tissue than NP cells in vitro and with the observed different mechanical properties of the engineered tissues using AF and NP cells. The results obtained thus far suggest that IVD tissues may be engineered in vitro using different cell sources (AF and NP) and that this process can be stimulated by growth factors such as OP-1.

TABLE 1
Characterization of Engineered Tissues

| | Culture | | |
|---|---|---|---|
| | Duration | AF disc | NP disc |
| Wet Weight | 2 w | 49.4 ± 1.7 | 132.7 ± 14.1** |
| (mg/tissue) | 4 w | 159.4 ± 6.0 | 166.9 ± 5.2* |
| Dry Weight | 2 w | 3.08 ± 0.09 | 7.21 ± 0.86* |
| (mg/tissue) | 4 w | 5.52 ± 0.09 | 7.34 ± 0.25*** |
| Water Content | 2 w | 93.8 ± 0.3 | 95.8 ± 0.1*** |
| (%) | 4 w | 94.1 ± 0.1 | 95.6 ± 0.1*** |
| Thickness | 2 w | 0.49 ± 0.13 | 1.37 ± 0.19*** |
| (mm) | 4 w | 0.60 ± 0.17 | 1.11 ± 0.57*** |
| DNA Content | 2 w | 5.26 ± 0.13 | 3.81 ± 0.05 |
| (μg/mg) | 4 w | 3.76 ± 0.13 | 3.48 ± 0.27 |
| Collagen Content | 2 w | 156.5 ± 5.1 | 92.2 ± 2.7 |
| (μg/mg) | 4 w | 188.7 ± 5.4 | 116.6 ± 3.2 |
| Proteoglycan | 2 w | 141.8 ± 1.0 | 112.2 ± 8.8 |
| Content (μg/mg) | 4 w | 117.7 ± 4.1 | 161.2 ± 4.3 |
| Hyaluronan | 2 w | 1.83 ± 0.23 | 2.09 ± 0.08 |
| Content (μg/mg) | 4 w | 2.61 ± 0.15 | 2.72 ± 0.25 |
| Crosslink Content | 2 w | 0.240 ± 0.006 | 0.596 ± 0.012 |
| (pmol/μg) | 4 w | 0.619 ± 0.036 | 1.001 ± 0.036 |
| H_{A0} (kPa) | 2 w | 2.85 ± 0.59 | 1.18 ± 0.27** |
| | 4 w | 2.08 ± 0.56 | 0.74 ± 0.43 |
| σₘₐₓ (mPa) | 2 w | 0.63 ± 0.05 | 0.33 ± 0.05 |
| | 4 w | 0.64 ± 0.06 | 0.23 ± 0.02 |
| log (kₚ₁₅) | 2 w | −13.5 ± 0.2 | −13.0 ± 0.3 |
| | 4 w | −13.0 ± 0.3 | −12.4 ± 0.4 |

(*p < 0.05, **p < 0.01, ***p < 0.005 versus AF)

### Example 2

### Transfection of Intervertebral Disc Cells with OP-1

### Cell and Tissue Preparation:

AF and NP tissues were isolated from IVD of tails of 14-18 month bovine steer. Cells were isolated by sequential digestion with 0.2% pronase and 0.025% collagenase+0.04% DNAase. The cells were seeded at a density of 50,000 cells/well in a 12-well plate and cultured for 2 days prior to transfection. After transfection the cells can be used to produce the tissue for transplantation as described in example 1. Alternatively, the tissues of IVD produced as in example 1 can be transfected using a gene gun. For tissues of IVD that were prepared for gene gun transfection, the cells and tissues were cultured in DMEM/F12 medium containing 10% FBS with a daily change of medium after the transfection.

### Reporter Gene and OP-1 Expression Vector

pCMV-β-galactosidase (Clontech) served as a reporter gene, and transgene expression was assessed using the in Situ β-galactosidase staining kit (Stratagene).

### Gene Transfer

At the time of gene transfer, a pulse of high pressure helium gas (125 psi) was released from a helium tank through Gold-Coat tubing, accelerating the DNA-coated gold particles on the inside of the tubing cartridge to penetrate the target cells. The gene gun was positioned at a minimal distance from the petri dish and tissue, and a single bombardment was carried out.

### Assessment of Transfection Efficiency

After 3 days, the transfection efficiency of a Lac reporter gene construct (pCMV-β-galactosidase) in the primary monolayer cultures of normal bovine NP and AF cells was assessed using an in Situ β-galactosidase staining kit.

### Measurement of Metabolic Activity of Transfected Cells

The DNA content and the total PG content were measured in the cell layer to assess metabolic activity. PG synthesis was also measured using ³⁵S-sulfate labeling, followed by rapid filtration and was compared between the OP-1-transfected (pW24) and the control (vacant vector) groups. Statistical analyses were performed by one-way ANOVA with Fisher's PLSD test as a post hoc test.

The gene transfer of β-galactosidase was performed to probe the efficiency of transfection in the three different cell sources. Analysis of X-gal staining demonstrated an efficiency of 10.1% in normal NP cells and 6.2% in AF cells (FIG. 2). The DNA content and rate of PG synthesis in the three cell types did not differ significantly when the pCMV-β-gal transfected and non-treated groups were compared. This suggested that the gene gun procedure does not have a significant adverse effect on cell metabolism.

To study whether gene transfection can alter the metabolism of IVD cells, the human OP-1 gene was transfected using a pW24 vector. On day 3 after transfection, there were no significant differences in the DNA content and PG content of the cell layer in any group. On the other hand, in the OP-1 transfected group, the rate of PG synthesis was significantly higher in all cell types [AF (124%) and NP (144%) cells (FIG. 3)] demonstrating NP cells were more responsive than AC and AF cells to the transfection of the OP-1 gene.

### Example 3

### Implantation of Engineered Intervertebral Tissue

Cells isolated from canine AF are cultured in alginate beads for up to 28 days. At various times, beads are removed and the matrix formed around the cells is analyzed as described below. In a second step, the cells with their cell-associated matrix (CM), are allowed to coalesce into a matrix as previously shown to be the case in Example 1.

For this experiment two mongrel dogs are sacrificed, for example by intravenous administration of excessive pentobarbital (Euthanasia B solution, Henry Schein Inc., Washington Port, N.Y.). Whole lumbar spines are harvested en bloc though a posterior mid-spinal incision under sterile conditions. As one example, lumbar discs (from L1-2 to L6-7) are immediately dissected and the NP and AF tissue separated by a blunt instrument. Cells are separately isolated from both AF and NP tissue by sequential enzymatic digestion at 37° C., such as in a humidified atmosphere of 5% CO₂ using 0.4% Pronase (Calbiochem, La Jolla, Calif.) for 1 hour followed by 0.025% collagenase-P (Boehringer Mannheim, Indianapolis, Ind.) and 0.04% deoxyribonuclease II (DNase II, Sigma Chemical Co., St. Louis, Mo.) overnight at 37° C. After digestion, the cells are washed, filtered such as through a 70 mm mesh (Becton Dickinson, Lincoln Park, N.J.) and counted, using a Coulter cell counter.

The isolated cells are resuspended in 1.2% low viscosity alginate solution (Keltone LV, Kelco) at 4 million cells/ml. Beads are formed by dispensing the alginate/cell suspension dropwise into a 102 mM CaCl₂ solution via a 22-gauge needle attached to a syringe pushed by a syringe pump at a known rate. After 10 minutes the newly formed beads (containing approximately 40,000 cells/bead) are washed with a sterile 0.9% saline solution followed by DMEM/F12. Beads containing AF cells are cultured in complete DMEM/F12 medium containing 10-20% FBS (HyClone, Logan, Utah), 200 ng/ml recombinant human osteogenic protein-1 (rhOP-1, Stryker Biotek), 25 mg/ml ascorbate and 10 mg/ml gentamicin in separate Petri dishes. The cultures are maintained with daily media changes for 7-28 days.

Nine beads are placed in each well of a 24-well plate (Corning Costar Corp., Cambridge, Mass.) and cultured in 0.4 ml of complete media containing one or other combinations and/or concentrations of growth factor (FBS, rhOP-1, FBS/rhOP-1) or no growth factor. The cultures are maintained for up to 28 days at 37° C. in a humidified atmosphere of 5% CO₂, with daily changes of media. On days 7, 14, 21 and 28, the medium is collected and beads dissolved by incubation for 20 minutes in 55 mM sodium citrate, 0.15M NaCl, pH 6.8, at 4° C. The cell-associated matrix and further removed matrix are separated by mild centrifugation for 10 min at 100 g, 4° C. The cell-associated matrix is enzymatically digested, such as at 56° C. for 24 hours in papain (20 mg/ml) (Sigma, St. Louis, Mo.), 0.1M sodium acetate, 50 mM EDTA, 5 mM cysteine hydrochloride, pH 5.53. After solubilization of the cells and their cell-associated matrix, the content of sulfated proteoglycan (PG) in the cell-associated matrix is measured by the dimethylmethylene blue (DMMB) method, and the content of DNA measured by the Hoechst 33258-dye method. Mok, S. S., et al., Aggrecan synthesized by mature bovine chondrocytes suspended in alginate: Identification of two distinct metabolic matrix pools. J Biol Chem, 1994. 269(52): p. 33021-7. A well established ELISA is used to measure the hyaluronan content, while the collagen content is quantified by reverse-phase high-performance liquid chromatography (RP-HPLC) after acid hydrolysis. Chiba, K., et al., Metabolism of the extracellular matrix formed by intervertebral disc cells cultured in alginate. Spine, 1997. 22(24): p. 2885-93. Collagen specific crosslinks (pyridinoline and deoxypyridinoline) are quantified using fluorescence detection following RP-HPLC. Petit, B., et al., Characterization of crosslinked collagens synthesized by mature articular chondrocytes cultured in alginate beads: comparison of two distinct matrix compartments. Exp Cell Res, 1996. 225(1): 151-61. The effect of culture duration (7, 14, 21 and 28 days) and growth factor (FBS, rhOP-1) treatments, and interaction between these treatments, is analyzed by 2-way ANOVA.

The present method is used to form intervertebral disc tissue in vitro. Annulus fibrosus cells are cultured in beads of alginate as described above. The cultures are kept for the optimal amount of time, after which, the beads are collected and dissolved as described above. The resulting suspension of cells (with their cell-associated matrix) is centrifuged and the pellet, containing the cells with their cell-associated matrix, is resuspended in complete medium. Cell culture plates (Falcon, Biocoat deep well, Becton Dickinson) are prewarmed for 20 minutes after addition of complete medium. Cell culture inserts (e.g., Falcon, PET membrane, 0.45 mm, 23 mm diameter, Becton Dickinson) are aseptically placed into each well of the culture plate, and a 5 ml aliquot of dissolved beads (corresponding to the cells and their cell-associated matrix present from 300 beads) plated onto each insert. The cultures are maintained at 37° C. in a humidified atmosphere of 5% CO₂.

To assess the biochemical composition of de novo formed tissue, the culture conditions are changed to provide a multiple number of samples using small inserts. The cells with their cell-associated matrix, recovered after the optimal time of culture in alginate (as determined above), are resuspended in complete medium and seeded onto a tissue culture insert as described above. After 7, 14, 21 and 28 additional days in culture on the insert, the de novo formed tissues are separated from the membrane and weights (wet and dry) measured. The tissues are examined histologically after staining with hematoxylin-eosin and Safranin-O/Fast green, and the contents of sulfated PG, DNA, hyaluronan and collagen are measured as described above. In addition, the matrix collagens are analyzed by SDS-PAGE and the profile of newly synthesized ³⁵S-PGs by dissociative CL-2B column chromatography (to assess phenotype). Hauselmann, H. J., et al., Phenotypic stability of bovine articular chondrocytes after long-term culture in alginate beads. J Cell Sci, 1994. 107(Pt 1): p. 17-27. The collagen types are assessed by immunohistochemistry using specific antibodies to canine types I and II collagen. The mRNA expression of types I and II (A & B) collagen and other extracellular matrix components is measured by real time PCR using a lightCycler (Roche Molecular Biochemicals, Indianapolis, Ind.) and the SYBRgreen Method. Ririe, K. M., R. P. Rasmussen, and C. T. Wittwer, Product differentiation by analysis of DNA melting curves during the polymerase chain reaction. Anal Biochem, 1997. 245(2): p. 154-60.

At the time points mentioned above, samples are prepared from tissue-engineered IVD tissue using a skin-biopsy punch. After sample isolation and immediately before testing, the thickness of each sample is measured using a contact-sensing micrometer at predetermined locations (e.g., 3 toward the edge, and 3 toward the center of the disc). The average of the measured specimen thickness is subsequently used. As a control, AF tissue from canine spine is prepared and analyzed. Compression tests are done on 9.6 mm diameter discs. The homogeneous confined compression properties are determined from static and dynamic tests [Chen, A., R. Schinagl, and R. Sah, Inhomogeneous and strain-dependent electromechanical properties of full-thickness articular cartilage. Trans Orthop Res Soc, 1998. 23: p. 225. Frank, E. H. and A. J. Grodzinsky, Cartilage electromechanics—II. A continuum model of cartilage electrokinetics and correlation with experiments. J Biomech, 1987. 20(6): p. 629-39. Lee, R. C., et al., Oscillatory compressional behavior of articular cartilage and its associated electromechanical properties. J Biomech Eng, 1981. 103(4): p. 280-92. Sah, R. L., S. B. Trippel, and A. J. Grodzinsky, Differential effects of serum, insulin-like growth factor-I, and fibroblast growth factor-2 on the maintenance of cartilage physical properties during long-termculture. J Orthop Res, 1996. 14(1): p. 44-52] using a conventional uniaxial mechanical tester (Dynastat, IMASS). Tensile tests are determined on IVD tissue strips (0.25 mm thick×2.5 mm wide, 8 mm long, tapered to 0.8 mm×4 mm in the gage region) sequentially cut from the tissue. The ends of the samples are secured in spring-loaded clamps, and extended by computer-controlled ramps to 5, 10, 15 and 20% stretch, with stress-relaxation to equilibrium at each stretch level. The equilibrium tensile modulus (Et) is calculated by fitting the approximate linear region (5-15% strain) of the stress-strain curve with the initial length taken as that corresponding to a stress of 0.01 Mpa. Masuda, K., et al. The alginate recovered-chondrocyte (ARC) method for the formation of cohesive cartilaginous tissue for articular cartilage repair. in International Symposium on Molecular Cell Biology of Cartilage Development and Repair, 1999. Matsumoto, T., et al. Tissue engineered intervertebral disc: enhancement of formation with osteogenic protein-1. in The transaction of International Conference Bone Morphogenetic Proteins, 2000. Chen, S., et al. Biomechanical properties of tissue-engineered cartilage synthesized using the ARC method. in International Symposium on Molecular Cell Biology of Cartilage Development and Repair. 1999. Guilak, F., et al., Mechanical and biochemical changes in the superficial zone of articular cartilage in canine experimental osteoarthritis. J Orthop Res, 1994. 12(4): p. 474-84. Setton, L. A., et al., Mechanical properties of canine articular cartilage are significantly altered following transection of the anterior cruciate ligament. J Orthop Res, 1994. 12(4): p. 451-63. Then, the sample is extended at a constant displacement rate (5 mm/min) until failure. The tensile strength (sult) is taken as the ultimate load normalized to the original cross-sectional area. These data allows selection of tissues with markedly different physicochemical properties (i.e. high and low tensile strength).

Having formed tissues with either low or high tensile strength, it is determined which of these tissues becomes most effectively integrated within the host when transplanted into a surgically created annular defect. The insertion of the tissue is randomized between the L2/3, L4/5, and L6/7 discs. The implant is in place for 3 or 30 days prior to euthanizing. Integration is assessed by short term cellular labeling, and histologically at the two time points.

The dog annulus defect model is established to obtain a full-defect in the AF. A total of 6 preconditioned mongrel dogs weighing 23-30 kg are used for this section of the experiment. All surgeries are performed under anesthesia and the surgical field is shaved and sterilized. After positioning the animal on the right lateral decubitus, the bony landmarks of the lumbar vertebral bodies are approached through a left-side retroperitoneal approach. The L2/3, L4/5, and L6/7 discs are exposed and confirmed, such as by radiographs. Then, a flap of the anterior longitudinal ligament is made at the disc space, and a defect in the postero-lateral portion of the AF (5 mm full depth into the NP) is created using a skin-biopsy punch (3 mm diameter). Extreme care is taken to protect the spinal cord and nerve roots, as they exist at each level. At the time of surgery, a titanium clamp is placed in the soft tissue to mark the incised IVD levels so that they can be identified subsequently. For the control discs, the defect is left empty. For the experimental discs, the tissue-engineered IVD tissue of either high or low tensile strength is transplanted in a randomized fashion. The tissue is rolled and inserted into the defect. Upon completion, the anterior longitudinal ligament flap is sutured to retain the tissue engineered IVD implant.

After each surgery, the wound is closed in anatomic layers and the dogs are allowed to recover from anesthesia in a humidified, warmed environment. After recovery, the dogs are returned to their cages. For analgesia, buprenorphine (0.01 mg/kg SQ b.i.d.) for 3 days and acetaminophen (0.3 g/kg PRN) can be administered. Kefzol (1 g 1M) can be given daily for 3 days prophylactically. Then the animals are housed separately to allow free ambulation and appropriate care. The staff veterinarian or a competent animal technician assesses animal health and general appearance on a daily basis. Deaths that occur outside of the schedule are recorded, and all of the necessary samples are retrieved immediately after death and processed per protocol for the histologic evaluations. Animals are sacrificed, for example with an IV injection of B-Euthanasia at 3 days and 30 days after the surgery.

Integration of engineered annulus into a defect is assessed by short term cellular labeling, and histology at the 3 day time point. IVD cells are labeled in vitro using 5-chlormethylfluorecein diacetate (CMFDA, Molecular Probes, Eugene, Oreg.) prior to insertion into the dog. Briefly, IVD cells are incubated in the presence of CMFDA prior to implantation into the annulus defect. After 3 days, the dogs are sacrificed, specimens collected, and processed.

To obtain a better understanding of the regeneration of the AF tissue in vivo, conventional histologic methods are used with attention given to the integrity of the transplanted and host tissues. Whole discs (L2/3, L4/5, and L6/7) are dissected from the study animals, and specimens corresponding to the implant area are isolated for processing. The specimens are fixed in 10% neutral buffered formalin for about one week. Decalcification at 37° C. is effected by placing the specimens in buffered (pH=7.0) 10% disodium ethylene diamine tetra-acetate (Na₂ EDTA) and 7.5% PVP-40 for 2-4 weeks; the decalcification is monitored using radiography. After decalcification, the specimens are rinsed in running tap water overnight, and placed in 70% ethyl alcohol. Next, all levels are dehydrated with graded alcohols, cleared in chloroform, and infiltrated with paraffin (melting point 56° C.). After infiltration with paraffin, the levels are oriented so that the mid-sagittal face is down and embedded in paraffin. The blocks are trimmed and sectioned on a rotary microtome with the section thickness alternating from 8 microns for conventional histology. In addition to conventional staining with Hematoxylin and Eosin, the sections are stained with Safranin-O/Fast Green to reveal areas rich in PGs, and the Gomori trichrome stain for fibrillar connective tissue. The zone of injury (fibrous tissue, fibrocartilage, or area of necrosis) is measured. Qualitative histological analysis is performed on the transplanted tissue-engineered tissue and the retention of the transplanted tissue in the host annulus tissue. Finally, both goals are combined in a preliminary electron microscopic analysis by carefully selecting interface regions of the transplanted engineered tissue (the transplanted tissue with the surrounding host tissue 1×2×4 mm). The specimens are trimmed, fixed further by immersion (overnight at 4° C.), rinsed in buffer, osmicated (2%), stained en bloc in uranyl acetate (1%), dehydrated, and infiltrated/embedded in epon/araldite. Semi-thin sections (1 mm) are stained with methylene blue/azure II for orientation and evaluation, followed by ultrathin sections doubly stained and examined at the ultrastructural level with the JEOL 100CX TEM.

To monitor the cell viability in engineered annulus fibrosus after implantation, the extent of apoptosis is assayed using a commercially available assay system that monitors DNA fragmentation (apoTac TM in situ, R&D Systems, Minneapolis, Minn.) on tissue sections. Carefully selected interface regions of the transplanted IVDs (the transplanted disc with the surrounding AF) are processed and sectioned for DNA fragmentation staining.

As will be understood by one skilled in the art, for any and all purposes, particularly in terms of providing a written description, all ranges disclosed also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “more than” and the like include the number recited and refer to ranges which can be subsequently broken down into subranges as discussed above. In the same manner, all ratios disclosed herein also include all subratios falling within the broader ratio.

One skilled in the art will also readily recognize that where members are grouped together in a common manner, such as in a Markush group, the present invention encompasses not only the entire group listed as a whole, but each member of the group individually and all possible subgroups of the main group. Accordingly, for all purposes, the present invention encompasses not only the main group, but also the main group absent one or more of the group members. The present invention also envisages the explicit exclusion of one or more of any of the group members in the claimed invention.

All references disclosed herein are specifically incorporated herein by reference thereto.

While preferred embodiments have been illustrated and described, it should be understood that changes and modifications can be made therein in accordance with ordinary skill in the art without departing from the invention in its broader aspects as defined in the following claims.

What is claimed is:

1. A method for producing an engineered intervertebral disc tissue, comprising:
(a) culturing intervertebral disc cells in a medium for an effective amount of time to produce intervertebral disc cells surrounded by a cell-associated matrix; and
(b) culturing the intervertebral disc cells surrounded by the cell-associated on a semipermeable membrane in the presence of one or more growth factors for a sufficient amount of time to produce a coherent, engineered intervertebral disc tissue.

2. The method of claim 1 further comprising one or more of:
(c) isolating the intervertebral disc cells prior to (a);
(d) recovering the intervertebral disc cells surrounded by the cell-associated matrix prior to (b);
(e) removing the engineered intervertebral disc tissue from the semipermeable membrane; or
(f) implanting the engineered intervertebral disc tissue into an in vivo intervertebral disc defect wherein the intervertebral disc tissue is implanted in the presence or absence of the semipermeable membrane.

3. The method of claim 1 wherein the intervertebral disc cells are nucleus pulposus or annulus fibrosus cells whereby an engineered nucleus pulposus tissue or engineered annulus fibrosus tissue is produced.

4. The method of claim 1 wherein the medium of (a) is an alginate medium

5. The method of claim 1 wherein the one or more growth factors is selected from the group consisting of osteogenic protein-1, bone morphogenetic proteins, cartilage-derived morphogenetic protein, platelet-derived growth factor, bone morphogenic protein-2, fibroblast growth factor, transforming growth factor beta, insulin-like growth factor and combinations thereof.

6. An engineered intervertebral disc tissue produced according to the method of claim 1.

7. The engineered intervertebral disc tissue of claim 6 wherein the tissue comprises collagen, hyaluronan, proteoglycan and water.

8. The engineered intervertebral disc tissue of claim 7 wherein a majority of the collagen comprises type I or type II.

9. A cohesive engineered intervertebral disc tissue comprised of greater than or about 80 percent water by weight, between at or about 0.95 and 7.5 μg/mg DNA, between at or about 100 and 350 μg/mg proteoglycan, and between at or about 75 and 450 μg/mg collagen, wherein the DNA, proteoglycan and collagen amounts are based on the dry weight of the engineered tissue.

10. The engineered intervertebral disc tissue of claim 9 further comprising between at or about 1.5 and 3.0 μg/mg hyaluronan based on the dry weight of the engineered intervertebral disc tissue.

11. The engineered intervertebral disc tissue of claim 9 wherein the DNA content of the tissue is between at or about 3 and 4.3 μg/mg, the proteoglycan content of the tissue is between at or about 100 and 200 μg/mg, the collagen content of the tissue is between at or about 75 and 175 μg/mg and further wherein a majority of the collagen is type II collagen.

12. The engineered intervertebral disc tissue of claim 9 wherein the DNA content of the tissue is between at or about 0.95 and 1.15 μg/mg, the proteoglycan content of the tissue is between at or about 275 and 350 μg/mg, the collagen content of the tissue is between at or about 350 and 450 μg/mg and further wherein a majority of the collagen is type II collagen.

13. The engineered intervertebral disc tissue of claim 9 wherein the DNA content of the tissue is between at or about 3.3 and 5.5 μg/mg, the proteoglycan content of the tissue is between at or about 100 and 185 μg/mg, the collagen content of the tissue is between at or about 125 and 250 μg/mg and further wherein a majority of the collagen is type I collagen.

14. A method for surgically repairing intervertebral disc damage, comprising:
(a) producing a transplantable intervertebral disc tissue in vitro; and
(b) implanting the intervertebral disc tissue into an intervertebral disc defect.

15. The method of claim 14 wherein (a) comprises:
(i) culturing intervertebral disc cells in a medium for an effective amount of time to produce intervertebral disc cells surrounded by a cell-associated matrix; and
(ii) culturing the intervertebral disc cells surrounded by the cell-associated on a semipermeable membrane in the presence of one or more growth factors for a sufficient amount of time to produce a coherent, engineered intervertebral disc tissue.

16. The method of claim 15 wherein (a) further comprises one or more of:
(iii) isolating the intervertebral disc cells prior to (i);
(iv) recovering the intervertebral disc cells surrounded by the cell-associated matrix prior to (ii); and
(v) removing the engineered intervertebral disc tissue from the semipermeable membrane.

17. The method of claim 15 wherein the intervertebral disc cells are annulus fibrosus cells and an annulus fibrosus tissue is produced or the intervertebral disc cells are nucleus pulposus tissue and a nucleus pulposus tissue is produced.

18. The method of claim 15 wherein the medium of (i) is an alginate medium.

19. The method of claim 15 wherein the one or more growth factors is selected from the group consisting of osteogenic protein-1, bone morphogenetic proteins, cartilage-derived morphogenetic protein, platelet-derived growth factor, bone morphogenic protein-2, fibroblast growth factor, transforming growth factor beta, insulin-like growth factor and combinations thereof.

20. A kit for producing an intervertebral disc tissue comprising:
(a) instructions for producing an intervertebral disc tissue; and one or more:
(b) growth media;
(c) semipermeable membranes;
(d) growth factors;
(e) one or more pieces of disposable lab equipment.
